(19) United States
(12) Patent Application Publication
(10) Pub. No.: US 2026/0125485 A1
(43) Pub. Date: May 7, 2026

(54) DENOSUMAB AND COLCHICINE TO PREVENT BLOOD CANCER AND AGING DISEASE IN PATIENTS WITH CLONAL HEMATOPOIESIS

(71) Applicant: VANDERBILT UNIVERSITY, Nashville, TN (US)

(72) Inventors: Taralynn MACK, Nashville, TN (US); Yash PERSHAD, Nashville, TN (US); Alexander BICK, Nashville, TN (US)

(21) Appl. No.: 19/370,908

(22) Filed: Oct. 28, 2025

Related U.S. Application Data
(60) Provisional application No. 63/715,157, filed on Nov. 1, 2024.

(51) Int. Cl. C07K 16/28; A61K 31/165; A61K 39/00; A61P 43/00

(52) U.S. Cl. CPC C07K 16/2875; A61K 31/165; A61P 43/00; A61K 2039/505; A61K 2039/545

(57) ABSTRACT

In one aspect, the disclosure relates to a method for treating clonal hematopoiesis of indeterminate potential (CHIP) in a subject, the method including at least the step of administering to the subject a therapeutically effective amount of an inhibitor of receptor activator of nuclear factor κB ligand (RANKL), such as denosumab, a therapeutically effective amount of an anti-inflammatory agent, such as colchicine, or both. In a further aspect, performing the method reduces growth of one or more clones or causes the size of one or more clones to remain constant. In another aspect, each month of performing the method can result in a further reduction of annual clonal growth rate. In a still further aspect, performing the method further treats or prevents a cardiometabolic disease, a cardiovascular disease, or a myeloproliferative disease or disorder. Also disclosed are methods for reducing or maintaining clonal hematopoiesis risk score (CHRS) in a subject.

### CROSS REFERENCE TO RELATED APPLICATIONS

This application claims the benefit of U.S. Provisional Application No. 63/715,157 filed on Nov. 1, 2024, which is incorporated herein by reference in its entirety.

### STATEMENT REGARDING FEDERALLY SPONSORED RESEARCH OR DEVELOPMENT

This invention was made with government support under grant OD029586 awarded by the National Institutes of Health. The government has certain rights in the invention.

### BACKGROUND

Clonal hematopoiesis of indeterminate potential (CHIP) occurs when a hematopoietic stem cell acquires a somatic driver mutation in a leukemia-associated gene, with a variant allele fraction (VAF) exceeding 2% in peripheral blood. Higher VAF is associated with increased morbidity and mortality risk. Identifying factors influencing clonal expansion rate is crucial for risk stratification in CHIP patients. Recent cost-effective targeted assays have enabled serial sequencing and longitudinal profiling of CHIP dynamics.

Despite advances in CHIP research, there is still a scarcity of compounds that are effective in the treatment and/or prevention of cardiovascular, cardiometabolic, and myeloproliferative diseases and disorders associated with CHIP. These needs and other needs are satisfied by the present disclosure.

### SUMMARY

In accordance with the purpose(s) of the present disclosure, as embodied and broadly described herein, the disclosure, in one aspect, relates to a method for treating clonal hematopoiesis of indeterminate potential (CHIP) in a subject, the method including at least the step of administering to the subject a therapeutically effective amount of an inhibitor of receptor activator of nuclear factor κB ligand (RANKL), such as denosumab, a therapeutically effective amount of an anti-inflammatory agent, such as colchicine, or both. In a further aspect, performing the method reduces growth of one or more clones or causes the size of one or more clones to remain constant. In another aspect, each month of performing the method can result in a further reduction of annual clonal growth rate. In a still further aspect, performing the method further treats or prevents at least one cardiometabolic disease, at least one cardiovascular disease, or at least one myeloproliferative disease or disorder. Also disclosed are methods for reducing or maintaining clonal hematopoiesis risk score (CHRS) in a subject.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1G show cohort characteristics and co-occurring clonal hematopoiesis of indeterminate potential (CHIP) mutations. (FIG. 1A) The cohort consists of 892 CHIP mutations in 711 individuals. Growth rate was calculated using a compound interest formula for sequencing at two blood draws. (FIG. 1B) Larger bar plot demonstrates the number of CHIP with a mutation in a driver gene. Smaller bar plot shows the number of individuals with 1, 2, 3, and 4 CHIP mutations. (FIG. 1C) Box plot of growth rate, calculated with a compound interest formula, for each CHIP mutation by driver gene with number of individuals with mutations in the driver gene shown below the gene name. Red diamond represents the mean growth rate. Box represents the interquartile range of the growth rate. Middle line in the box represents the median of the growth rate. (FIG. 1D) Theoretical example of possible trajectories for individuals with two CHIP mutations. The mutations can either be in distinct cell populations or the same cell population. Variant allele fraction (VAF) trajectories for each of these scenarios should follow similar trends to this example. Each mutation is represented by color, and the line represents change in VAF between first and second blood draws. (FIG. 1E) Box plot showing the difference in average growth rate of the fastest growing mutations for the 116 individuals grouped into the distinct vs sub-clone categories. The red diamond represents the average of the growth rate. Box represents the interquartile range of the growth rate. Middle line in the box represents the median of the growth rate. The x-axis is the growth rate while the y-axis is the category. (FIG. 1F) Upset plot showing the different combinations of driver genes represented within the group of 116 individuals with multiple CHIP mutations. Above the upset plot is a bar plot showing the distribution between distinct vs sub-clone trajectories for each of the driver gene combinations, with driver gene on the x-axis and count on the y-axis. (FIG. 1G) Heat map showing the deviation from random for the co-occurrence of each driver gene pair for the 116 individuals with 2 CHIP mutations. Each gene pair was tested with a Fisher's exact test to identify deviation from expected occurrence via random chance. The x and y-axes represent each gene in a pair, the color of the box represents the direction of the deviation from random, and the darkness of the hue represents the magnitude of the deviation. For gene combinations with a p-value >0.05, the odds ratio is displayed, and the box is marked with “*” and for gene combinations that passed multiple-hypothesis correction with a p-value of <0.001, the odds ratio is displayed and the box is marked with “**”.

FIGS. 2A-2E show determinants of clonal hematopoiesis of indeterminate potential (CHIP) growth rate. (FIG. 2A) Forest plot of change in annual growth rate (%/year) with each additional copy of the alternate allele (alt allele) for select germline single nucleotide polymorphisms (SNPs) detectable with the targeted sequencing assay among individuals with CHIP mutations. Effect estimate represents the coefficient of a linear regression of growth rate and number of alternate alleles modeled additively as 0, 1, or 2, adjusting for age, sex, race, and variant allele fraction at first sequencing. The 95% confidence interval (95% CI) was computed as the Effect Estimate±1.96×Standard Error. The p-value tests the null hypothesis of the effect estimate being 0. (FIG. 2B) Violin plot of annual growth (%/year) for 0 versus >1 G allele for germline variant chr11:108272729:C:G in ATM. When modeled additively, each additional G allele is associated with greater annual growth rate (%/year) in multiple linear regression by 0.46% per year (95% CI: 0.23 to 0.67, P<0.001). There were 619 CHIP mutations in individuals with 0 G alleles, 26 in individuals with 1 G allele, and 2 in individuals with 2 G alleles. (FIG. 2C) Violin plot of annual growth (%/year) for 0, 1, and 2 T alleles for germline variant chr14:95714358:G:T in TCL1A for DNMT3A CHIP (left) and TET2 CHIP (right). When modeled additively, each additional T allele is associated with greater annual growth rate (%/year) for TET2 CHIP mutations (P=0.03), but not for DNMT3A CHIP mutations (P=0.49). There were 132 TET2 mutations in individuals with 0 T alleles, 54 TET2 mutations in individuals with 1 T allele, and 6 TET2 mutations with 2 T alleles. There were 158 DNMT3A mutations in individuals with 0 T alleles, 95 DNMT3A mutations in individuals with 1 T allele, and 28 DNMT3A mutations in individuals with 2 T alleles. (FIG. 2D) Forest plot of change in annual growth rate (%/year) with each additional prescription month of 4 select drugs with suggestive associations: colchicine, denosumab, methylprednisolone, and hydroxychloroquine. Black effect estimate represents the coefficient of a linear regression of growth rate and number of months exposed to the drug adjusting for age, sex, race, and variant allele fraction at first sequencing with all data. Gray effect estimate represents the coefficient of a linear regression of growth rate and number of months exposed to the drug for 3:1 matched non-drug-exposed controls to drug-exposed cases (matched by age, sex, CHIP driver gene, variant allele fraction at first sequencing). The 95% confidence interval (95% CI) was computed as the Effect Estimate±1.96×Standard Error. The p-value tests the null hypothesis of the effect estimate being 0. N represents the number of individuals prescribed the drug for at least 1 month. (FIG. 2E) Forest plot of change in annual growth rate (%/year) among individuals with select pre-existing diagnoses based on phecodes. Individuals had to have their first diagnosis of the phecode before their second blood draw. Estimate represents the coefficient of a linear regression of growth rate and presence of diagnosis as a binary variable adjusting for age, sex, race, and variant allele fraction at first sequencing. The 95% confidence interval (95% CI) was computed as the Effect Estimate±1.96×Standard Error. The p-value tests the null hypothesis of the effect estimate being 0. N represents the number of individuals with the diagnosis in each regression.

FIGS. 3A-3D show phenotypic consequences of growth rate. (FIG. 3A) On the left, Kaplan-Meier curve of time to low myeloid counts-defined as thrombocytopenia (platelet count <169.06×10⁹ cells/L), anemia (red blood cell count <3.96×10¹² cells/L) or neutropenia (neutrophil count <1.47×10⁹ cells/L) for individuals with a CHIP growth rate >16% annually (red) and <16% annually (gray). A Cox proportional hazard model of time to low myeloid counts and rank-inverse normalized growth rate, when adjusting for age, variant allele fraction (VAF) at the first blood draw, and sex was significant (HR=1.20, 95% CI 1.05-1.36, P<0.001). On the right, Kaplan-Meier curve of time to high myeloid counts-defined as thrombocytosis (platelet count >397.1×10⁹ cells/L), polycythemia (red blood cell counts >5.5×10¹² cells/L), or elevated neutrophil count (neutrophil count >7.06×10⁹ cells/L) for individuals with a CHIP growth rate >16% annually (red) and <16% annually (gray). A Cox proportional hazard model of time to high myeloid counts and rank-inverse normalized growth rate, when adjusting for age, VAF at the first blood draw, and sex was significant (HR=1.14, 95% CI 1.02-1.27, P=0.003). (FIG. 3B) Forest plot showing the association between growth rate and time to event for the listed phenotypes. Out of the 711 individuals in the study, individuals who had a phenotype for the first time after the second blood draw are included, with each phenotype sample size listed in the figure. (FIG. 3C) Heatmap showing Clonal Hematopoiesis Risk Score (CHRS) category at the first blood draw (TP1) versus risk at second blood draw (TP2) for N=134 individuals with data available to compute a CHRS (blood counts, mean corpuscular volume, and red cell distribution width). As per Weeks et al, low risk was defined as CHRS≤9.5, intermediate risk as 10<CHRS<12, and high risk as CHRS ≥12.5. Darker hues represent higher numbers of individuals, and exact counts are displayed for each category. (FIG. 3D) Heatmap showing the change in each of the CHRS criteria for the N=30 individuals that shifted into a different risk category between first and second blood draw. The x-axis represents each individual and the y-axis represents the CHRS components, which are faceted based on what clinical test would need to be performed to ascertain that information (i.e., none, blood panel, and sequencing). The color of the box represents the direction of the change (red is positive and blue is negative) and the darkness of the hue represents the magnitude of the change.

FIG. 4 shows a flow chart representing patient selection criteria for inclusion in the study. CHIP=Clonal hematopoiesis of indeterminate potential. MTP=multiple time point. VAF=variant allele fraction. Mack et al, 2024 is reference 6 in the manuscript.

FIG. 5A shows clonal trajectories of 6 clones in 4 individuals with multiple sequencing blood draws while taking colchicine plotting variant allele frequency (VAF) over time in months from the Clonal Hematopoiesis and Inflammation in the VasculaturE (CHIVE) cohort. Green dotted line represents the starting of colchicine. Red dotted line represents the discontinuation of colchicine. Patient ID and CHIP driver gene is shown in each subfigure title. FIG. 5B shows clonal trajectories of 10 clones in 5 individuals with multiple sequencing blood draws while taking oral methylprednisolone plotting variant allele frequency (VAF) over time in months from the Clonal Hematopoiesis and Inflammation in the VasculaturE (CHIVE) cohort. Green dotted line represents the starting of methylprednisolone. Red dotted line represents the discontinuation of methylprednisolone. Patient ID and CHIP driver gene is shown in each subfigure title.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Aging is associated with the accumulation of somatic mutations across cells. Similar to other stem cells, hematopoietic stem cells (HSCs) accumulate mutations leading to increasing genetic diversity across an individual's lifetime. Individual HSCs are estimated to acquire 200 mutations per decade genome-wide, with 1 mutation per decade occurring within an exonic region. While the vast majority of such mutations do not have substantive impacts on cellular fitness, occasionally one such mutation may promote vitality and proliferation termed clonal hematopoiesis (CH).

CH has long been hypothesized as a key precursor in a sequential model of leukemogenesis. Age-related HSC clonal abnormalities in asymptomatic individuals was first recognized three decades ago through the analyses of non-random X-inactivation patterns derived from peripheral leukocytes of women. Population-based next-generation sequencing over the last decade has shown that CH is surprisingly common with approximately 1 in 10 asymptomatic adults older than 70 years affected. Using whole exome sequences of blood DNA originally aimed to discover rare germline disruptive coding alleles contributing to risk for common complex diseases, investigators employed methods to detect acquired mutations. ‘Clonal hematopoiesis of indeterminate potential’ (CHIP) is the presence of a hematologic malignancy driver mutation (typically in DNMT3A, TET2, ASXL1, JAK2) with high variant allele frequency in blood (i.e., >2%) indicative of clonality. While CHIP is a strong risk factor for hematologic malignancy, risk is not absolute with ˜0.5%/year progression from CHIP to hematologic malignancy.

A more surprising finding related to CHIP is that its implications for coronary artery disease may be a more important than hematologic malignancy. In several datasets, CHIP is associated with a 1.6-1.9-fold risk for coronary artery disease (CAD), and thus larger absolute risk increase for CAD compared to hematologic malignancy. Among asymptomatic individuals, individuals with CHIP have a greater burden of subclinical coronary atherosclerosis compared to those without. Consistent with the human observations, irradiated mice transplanted with Tet2−/− bone marrow versus transplanted with wild type bone marrow have a greater burden of supravalvular and descending aortic atherosclerosis. Both humans and mice with CHIP mutations in hematopoietic stem cells have greater concentrations of circulating inflammatory cytokines. Inhibition of the NLRP3 inflammasome mitigates atherosclerosis to a greater degree in irradiated mice transplanted with Tet2−/− bone marrow versus transplanted with wild type bone marrow. Similarly, genetic deficiency of IL6-receptor, in the NLRP3 pathway, through the presence of a common IL6R missense mutation in humans is associated with a greater reduction in cardiovascular disease risk among those with CHIP versus without. These data imply that for patients with CHIP, a tailored anti-inflammatory approach may be highly effective at addressing CHIP-associated cardiovascular disease risk. The increasingly robust therapeutic hypothesis is ripe for testing in placebo-controlled clinical trials.

Additional forms of CH have also been detected from the analysis of blood DNA. Larger chromosomal rearrangements, often term mosaic chromosomal alterations (mCAs) or clonal somatic copy number alterations, have been identified from large-scale blood DNA-derived genome-wide genotyping. While CHIP is strongly associated with myeloid malignancies, mCAs are strongly associated with lymphoid malignancies. Unlike CHIP, mCAs are not associated with CAD. Additionally, mCAs may represent more widespread immunologic dysfunction as they predict diverse incident cancers and infections.

Clonal hematopoiesis of indeterminate potential, or CHIP, is a common aging-related phenomenon in which hematopoietic stem cells (HSCs) or other early blood cell progenitors contribute to the formation of a genetically distinct subpopulation of blood cells. As the name suggests, this subpopulation in the blood is characterized by a shared unique mutation in the cells' DNA; it is thought that this subpopulation is “clonally” derived from a single founding cell and is therefore made of genetic “clones” of the founder.

Clonal hematopoiesis by itself is not considered to be a hematologic cancer; nevertheless, evidence is mounting that this condition may adversely affect human health. It has been proposed to label the group of individuals who have clonal hematopoiesis defined by a mutation in a malignancy-associated gene but without evidence of disease (such as cytopenia, dysplasia or immature “blast” cells in the bone marrow) as having CHIP. A clonal involvement (sometimes referred to simply as the size of a “clone”) of 2% of the blood has been tentatively proposed as a cutoff, though there is discussion that a lower floor that is more inclusive could also be appropriate. This cutoff may ultimately depend on whether clones must reach a certain size before influencing health. The level at which a clone begins to have a potential clinical impact is an open question, though there is already data to suggest larger clones have a larger effect on health.

The presence of clonal hematopoiesis/CHIP has been shown to increase blood cancer risk and is correlated with an increased risk of mortality overall. This is true both of clonal hematopoiesis with known candidate drivers as well as in cases without such drivers.

One area of health that CHIP has been definitively shown to influence is the risk of progression to blood cancer. In a given year, a tiny fraction of the general population will develop a hematologic cancer such as myelodysplastic syndrome (MDS) or AML; it is estimated that just 3 to 4 people per 100,000 will get MDS in a given year, and 4 people per 100,000 will develop AML. With CHIP, the risk of acquiring a hematologic malignancy like MDS or AML is increased more than 10-fold. Despite this increased risk, people with CHIP are still at low overall risk for developing a blood cancer, with only about 0.5-1.0% transformation per year.

A second area of health that may be affected by CHIP is the risk for heart attack and stroke. A strong association between CHIP and heart attack/ischemic stroke has been identified in multiple human genetic datasets, where CHIP was a stronger predictor of heart attack/stroke than if a patient 1) was a smoker, 2) had hypertension, 3) had high cholesterol, or 4) was overweight. In this study, which shows correlation but not causation, people with CHIP were 2.3 times more likely to suffer a heart attack, or 4.4 times as likely if the variant allele frequency in their blood was greater than 0.10, than matched controls without CHIP. It has also been found that there is an increased risk of cardiovascular mortality in patients who exhibit CHIP and receive self-derived stem cell transplantation. The idea of CHIP having a causal role in human heart attacks/strokes has been given support by a 2017 study that showed impairment of the Tet2 CHIP gene in mice causally led to accelerated atherosclerosis, and this finding in mice has been independently validated. The possibility of somatic mutations in the blood contributing not only to cancer risk but also to heart attack and stroke has generated much discussion in top-level scientific publications and a large multi-cohort study published in 2017 appears to confirm the causal link between CHIP and cardiovascular disease in humans.

In addition to its effects on those who would otherwise be considered healthy, CHIP may have implications in certain disease contexts. It has been shown that patients with CHIP who receive autologous stem cell transplantation (ASCT) as part of their treatment for lymphoma have worse outcomes than patients without CHIP. The poorer prognosis for these patients is due to both an increase in subsequent therapy-related myeloid neoplasms and increased risk for cardiovascular mortality.

Disclosed herein is a method for treating clonal hematopoiesis of indeterminate potential (CHIP) in a subject, the method including at least the step of administering to the subject a therapeutically effective amount of an inhibitor of receptor activator of nuclear factor κB ligand (RANKL), a therapeutically effective amount of an anti-inflammatory agent, or both.

In one aspect, the inhibitor of RANKL can be denosumab. In another aspect, the inhibitor of RANKL can be administered by injection. In still another aspect, the inhibitor of RANKL can be administered once every six months.

In a further aspect, the anti-inflammatory agent can be colchicine. In another aspect, the anti-inflammatory agent can be administered orally. In still another aspect, the anti-inflammatory agent is administered daily.

In any of these aspects, the method can further include detecting at least one CHIP mutation in the subject prior to performing the method. In another aspect, performing the method reduces growth of one or more clones relative to an untreated control. In another aspect, performing the method causes a size of one or more clones to remain constant.

In some aspects, the method is performed for at least one year. Further in this aspect, each month of performing the method results in a reduction of annual clonal growth rate of from about 2.5% to about 3.5%.

In one aspect, both an inhibitor of RANKL and an anti-inflammatory agent are administered to the subject.

In another aspect, performing the method further treats or prevents at least one cardiometabolic or cardiovascular disease, such as, for example, peripheral artery disease, non-alcoholic fatty liver disease (NAFLD), hypertension, hyperlipidemia, or atherosclerosis. In still another aspect, performing the method further treats or prevents at least one myeloproliferative disease or disorder such as, for example, chronic myelomonocytic leukemia, chronic myeloid leukemia, anemia, thrombocytopenia, thrombocytosis, polycythemia, or any combination thereof. In some embodiments, the cardiometabolic disease is atherosclerosis, coronary heart disease (CHD) or ischemic stroke (IS). In some embodiments, the hematological cancer is a leukemia, a lymphoma, a myeloma or a blood syndrome. In some embodiments, the leukemia is acute myeloid leukemia (AML) or chronic myelogenous leukemia (CML). In some embodiments, the blood syndrome is myelodysplastic syndrome (MDS).

Also disclosed herein is a method for reducing or maintaining clonal hematopoiesis risk score (CHRS) in a subject relative to the same subject at an earlier point in time, the method including at least the step of administering to the subject a therapeutically effective amount of an inhibitor of receptor activator of nuclear factor κB ligand (RANKL), a therapeutically effective amount of an anti-inflammatory agent, or both. Further in this aspect, the inhibitor of RANKL can be denosumab and the anti-inflammatory agent can be colchicine.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a RANKL inhibitor,” “a clone,” or “an anti-inflammatory agent,” include, but are not limited to, mixtures, combinations, or populations of two or more such RANKL inhibitors, clones, or anti-inflammatory agents, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a therapeutic agent (e.g., RANKL inhibitor or anti-inflammatory agent) refers to an amount that is sufficient to achieve the desired improvement in the property modulated by the formulation component, e.g. achieving a decrease in clone size or maintaining a constant clone size. The specific level in terms of dosage required as an effective amount will depend upon a variety of factors including the age, sex, and body weight of the subject, clonal hematopoiesis risk score, other medications being taken by the subject, and diseases and conditions with which the subject has already been diagnosed.

As used herein, the terms “optional” or “optionally” means that the subsequently described event or circumstance can or cannot occur, and that the description includes instances where said event or circumstance occurs and instances where it does not.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The compositions disclosed herein may be administered prophylactically to patients or subjects who are at risk for the disease. Thus, the method can further comprise identifying a subject at risk for the disease prior to administration of the herein disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Sequencing

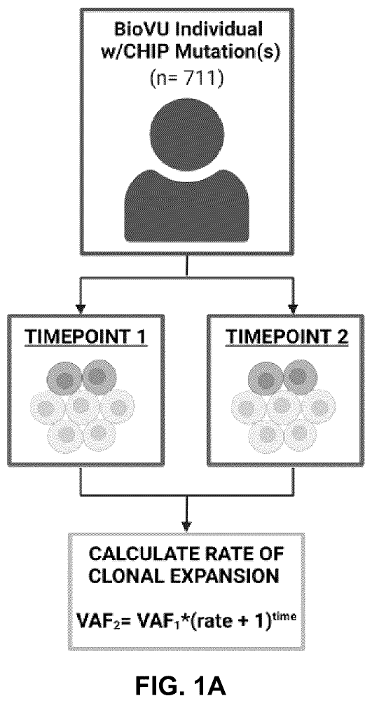

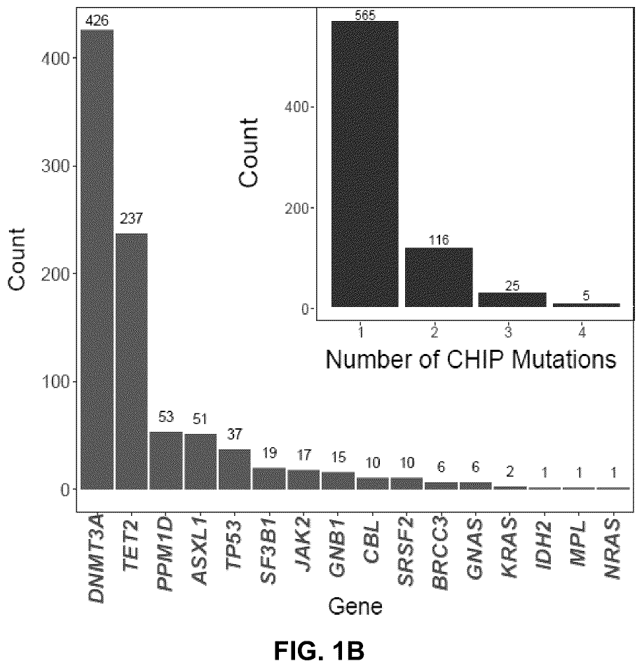

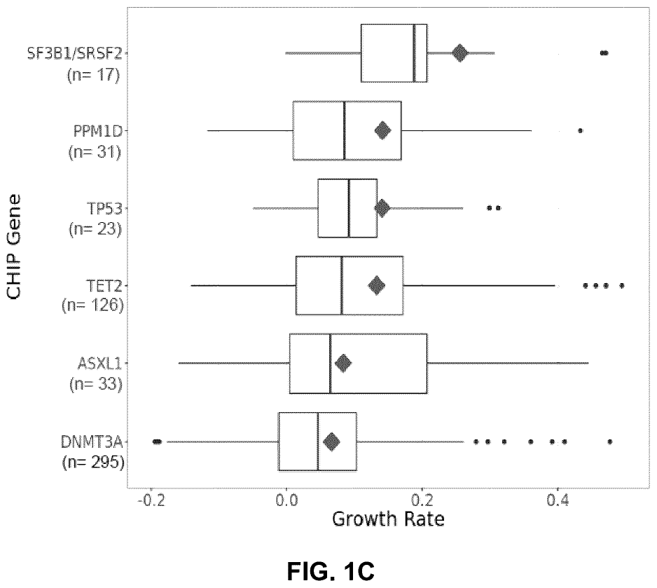

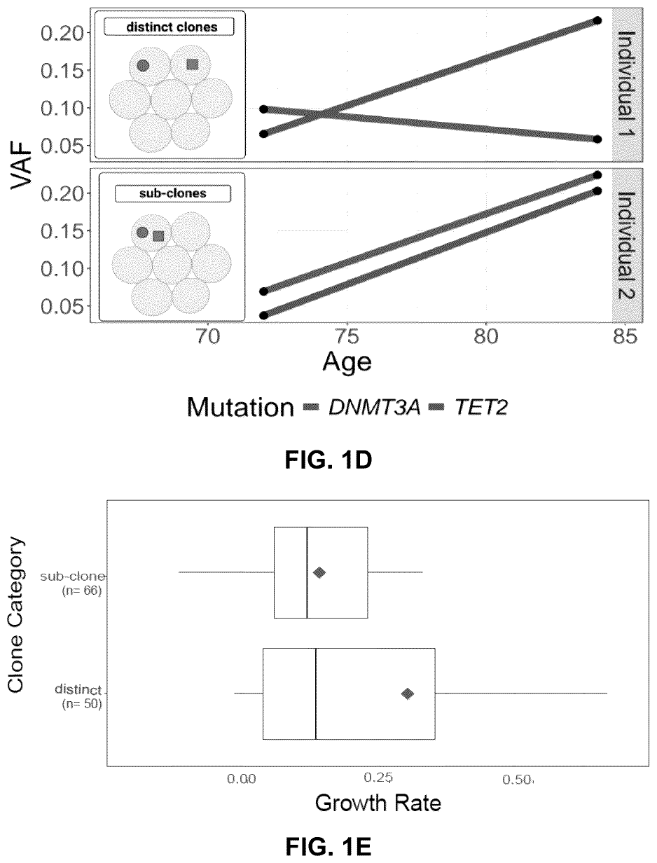

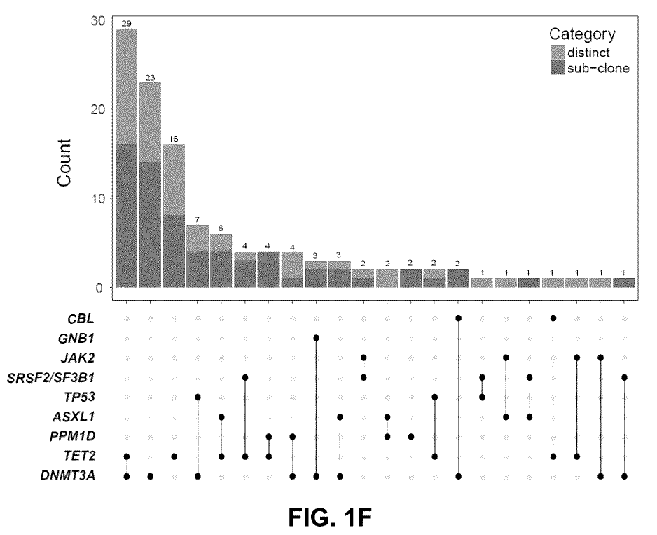

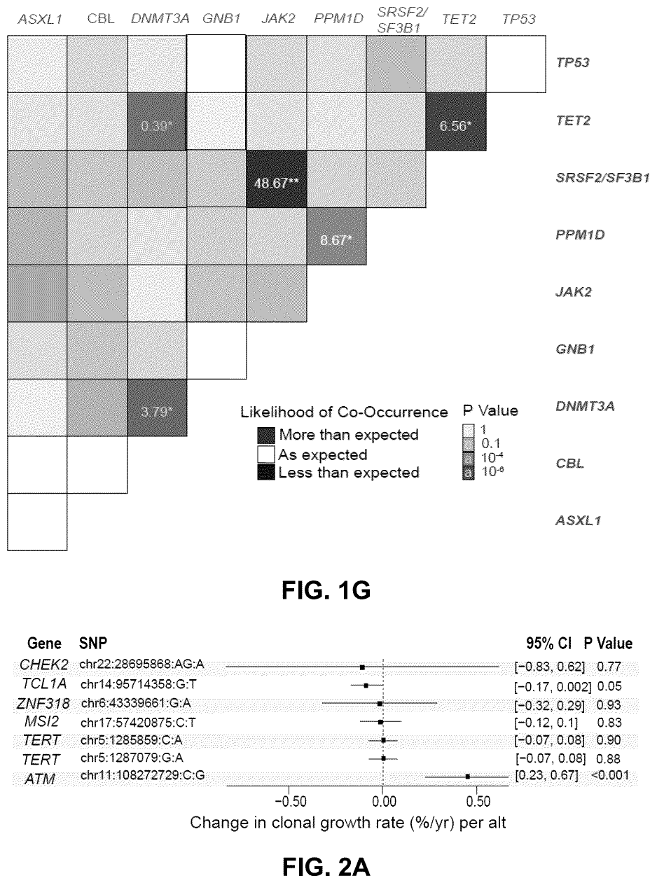

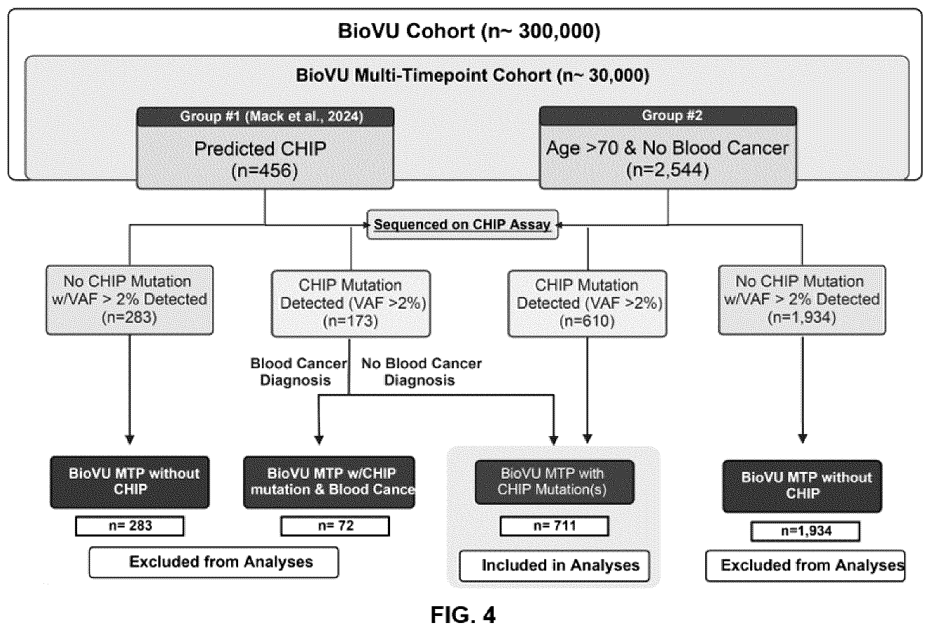

Targeted, error-corrected sequencing was performed on serial blood samples from 3,000 individuals in the Vanderbilt BioVU biobank, using custom-designed probes for 22 CHIP-associated genes (Table 1; FIG. 4). Vanderbilt University Medical Center's Institutional Review Board oversees BioVU and approved this project (IRB #201783). Unique molecular identifiers (UMI) were used for error correction, excluding mutations detected from a single UMI. The mean coverage depth was 1725× after de-duplication. CHIP mutations were called for variants with ≥100× total read depth, ≥3 variant allele reads, and >2% VAF in at least one blood draw. 893 CHIP mutations were identified in 711 individuals (FIG. 1A). The mean age of the participants at the first blood draw was 70 (range: 19-96). Participants' mean age at first draw was 70 years (range: 19-96), with a mean 5.7-year interval (range: 0.7-13) between samples. Mean VAFs were 6.7% and 9.5% at first and second draws, respectively. Most individuals (79%) had a single CHIP mutation, 16% had two, and 4% had three or more (FIG. 1B). DNMT3A and TET2 were the most frequently mutated genes. Of the 711 individuals, 74% had CHIP at both timepoints, while 26% had >2% VAF at only one draw, predominantly (78%) at the second draw.

TABLE 1
Targeted Capture regions for identifying a CHIP mutation. Genomic
coordinates are provided per the hg38 reference genome.

| Chromosome | Start | Stop | Gene |
|---|---|---|---|
| chr1 | 1806469 | 1806543 | GNB1 |
| chr1 | 1815750 | 1815867 | GNB1 |
| chr1 | 43349257 | 43349363 | MPL |
| chr1 | 114713799 | 114713978 | NRAS |
| chr1 | 114716047 | 114716162 | NRAS |
| chr2 | 25749689 | 25750420 | ASXL2 |
| chr2 | 25753532 | 25753640 | ASXL2 |
| chr2 | 25246614 | 25246781 | DNMT3A |
| chr2 | 25313907 | 25313989 | DNMT3A |
| chr2 | 25234273 | 25234425 | DNMT3A |
| chr2 | 25235701 | 25235830 | DNMT3A |
| chr2 | 25236930 | 25237010 | DNMT3A |
| chr2 | 25239124 | 25239220 | DNMT3A |
| chr2 | 25239484 | 25239518 | DNMT3A |
| chr2 | 25240296 | 25240455 | DNMT3A |
| chr2 | 25240634 | 25240735 | DNMT3A |
| chr2 | 25241556 | 25241712 | DNMT3A |
| chr2 | 25243892 | 25243987 | DNMT3A |
| chr2 | 25244149 | 25244343 | DNMT3A |
| chr2 | 25244534 | 25244657 | DNMT3A |
| chr2 | 25245247 | 25245337 | DNMT3A |
| chr2 | 25246014 | 25246069 | DNMT3A |
| chr2 | 25246154 | 25246314 | DNMT3A |
| chr2 | 25247045 | 25247163 | DNMT3A |
| chr2 | 25247585 | 25247754 | DNMT3A |
| chr2 | 25248031 | 25248257 | DNMT3A |
| chr2 | 25249651 | 25249729 | DNMT3A |
| chr2 | 25251906 | 25252099 | DNMT3A |
| chr2 | 25252188 | 25252202 | DNMT3A |
| chr2 | 25274935 | 25275092 | DNMT3A |
| chr2 | 25275494 | 25275548 | DNMT3A |
| chr2 | 25282382 | 25282716 | DNMT3A |
| chr2 | 25300133 | 25300248 | DNMT3A |
| chr2 | 208243524 | 208243601 | IDH1 |
| chr2 | 208248358 | 208248421 | IDH1 |
| chr2 | 197400709 | 197400941 | SF3B1 |
| chr2 | 197401979 | 197402135 | SF3B1 |
| chr2 | 197405269 | 197405477 | SF3B1 |
| chr2 | 197416735 | 197416916 | SF3B1 |
| chr2 | 197400049 | 197400171 | SF3B1 |
| chr2 | 197400246 | 197400439 | SF3B1 |
| chr2 | 197401394 | 197401530 | SF3B1 |
| chr2 | 197401736 | 197401893 | SF3B1 |
| chr2 | 197402550 | 197402831 | SF3B1 |
| chr2 | 197402943 | 197403040 | SF3B1 |
| chr2 | 197403579 | 197403769 | SF3B1 |
| chr2 | 197405070 | 197405182 | SF3B1 |
| chr2 | 197407992 | 197408124 | SF3B1 |
| chr2 | 197408363 | 197408586 | SF3B1 |
| chr2 | 197409764 | 197410012 | SF3B1 |
| chr4 | 54727217 | 54727324 | KIT |
| chr4 | 54733069 | 54733192 | KIT |
| chr4 | 54727415 | 54727542 | KIT |
| chr4 | 54727822 | 54727927 | KIT |
| chr4 | 54728010 | 54728121 | KIT |
| chr4 | 54729334 | 54729485 | KIT |
| chr4 | 54731327 | 54731419 | KIT |
| chr4 | 54731870 | 54731998 | KIT |
| chr4 | 54736497 | 54736609 | KIT |
| chr4 | 105233891 | 105237445 | TET2 |
| chr4 | 105243564 | 105243783 | TET2 |
| chr4 | 105269604 | 105269752 | TET2 |
| chr4 | 105275042 | 105276524 | TET2 |
| chr4 | 105241333 | 105241438 | TET2 |
| chr4 | 105242828 | 105242932 | TET2 |
| chr4 | 105259613 | 105259774 | TET2 |
| chr4 | 105261753 | 105261853 | TET2 |
| chr4 | 105272558 | 105272923 | TET2 |
| chr9 | 5076681 | 5076701 | JAK2 |
| chr9 | 5073683 | 5073800 | JAK2 |
| chr11 | 119278160 | 119278302 | CBL |
| chr11 | 119278504 | 119278718 | CBL |
| chr12 | 22671265 | 22671359 | ETNK1 |
| chr12 | 25225612 | 25225772 | KRAS |
| chr12 | 25227220 | 25227424 | KRAS |
| chr12 | 25245270 | 25245384 | KRAS |
| chr15 | 90088655 | 90088758 | IDH2 |
| chr17 | 60656593 | 60656846 | PPM1D |
| chr17 | 60662989 | 60663557 | PPM1D |
| chr17 | 7669603 | 7669695 | TP53 |
| chr17 | 7670603 | 7670720 | TP53 |
| chr17 | 7673213 | 7673271 | TP53 |
| chr17 | 7673301 | 7673344 | TP53 |
| chr17 | 7673529 | 7673613 | TP53 |
| chr17 | 7673695 | 7673842 | TP53 |
| chr17 | 7674175 | 7674295 | TP53 |
| chr17 | 7674814 | 7674976 | TP53 |
| chr17 | 7675047 | 7675243 | TP53 |
| chr17 | 7675988 | 7676277 | TP53 |
| chr17 | 7676376 | 7676408 | TP53 |
| chr17 | 7676515 | 7676627 | TP53 |
| chr18 | 44951903 | 44952002 | SETBP1 |
| chr20 | 32358770 | 32358837 | ASXL1 |
| chr20 | 32359741 | 32359796 | ASXL1 |
| chr20 | 32366378 | 32366472 | ASXL1 |
| chr20 | 32369006 | 32369128 | ASXL1 |
| chr20 | 32428122 | 32428253 | ASXL1 |
| chr20 | 32428319 | 32428427 | ASXL1 |
| chr20 | 32429332 | 32429436 | ASXL1 |
| chr20 | 32429895 | 32430058 | ASXL1 |
| chr20 | 32431315 | 32431489 | ASXL1 |
| chr20 | 32431577 | 32431684 | ASXL1 |
| chr20 | 32432874 | 32432990 | ASXL1 |
| chr20 | 32433278 | 32433922 | ASXL1 |
| chr20 | 32434426 | 32437343 | ASXL1 |
| chr20 | 58910677 | 58910834 | GNAS |
| chr20 | 58909344 | 58909428 | GNAS |
| chr20 | 58909515 | 58909584 | GNAS |
| chr20 | 58909678 | 58909809 | GNAS |
| chr20 | 58909945 | 58910086 | GNAS |
| chr20 | 58910328 | 58910406 | GNAS |
| chr21 | 43094649 | 43094793 | U2AF1 |
| chr21 | 43093096 | 43093254 | U2AF1 |
| chr21 | 43094461 | 43094568 | U2AF1 |
| chr21 | 43095432 | 43095541 | U2AF1 |
| chr21 | 43095688 | 43095748 | U2AF1 |
| chr21 | 43100447 | 43100524 | U2AF1 |
| chr21 | 43101274 | 43101437 | U2AF1 |
| chr21 | 43104309 | 43104407 | U2AF1 |
| chr21 | 43107445 | 43107499 | U2AF1 |
| chrX | 155071527 | 155071650 | BRCC3 |
| chrX | 155072326 | 155072343 | BRCC3 |

### Example 2: Growth Rate Modeling

The growth rate r was modeled with a compound interest formula

$$r = \frac{{VAF}_{2}^{1 / time}}{{VAF}_{1}} - 1.$$

SRSF2/SF3B1 (splicing factor) driver mutations exhibited the fastest average growth rate (˜25% annually), while DNMT3A driver mutations showed the slowest rate (˜7% annually) (FIG. 1C). 78% of mutations increased in VAF (annual growth rate >1%), consistent with prior studies. Decreases in VAF (annual growth rate <−1%) were observed in 30% of JAK2, 21% of ASXL1, 25% of DNMT3A, 19% of PPM1D, 19% of TET2, and 0% of SRSF2/SF3B1 mutations. While certain driver genes are more prone to expansion, CHIP expansion rates varied considerably among individuals with the same driver gene. This variability may necessitate larger sample sizes in prospective randomized controlled trials aiming to identify drugs that slow expansion.

### Example 3: Mutation Pairs

Individuals with two CHIP mutations, which may co-occur as subclones or in distinct cells, were analyzed (FIG. 1D). Mutation pairs were classified as subclones if their growth rates were within 0.6 standard deviations of each other; otherwise, they were considered distinct. Of 116 individuals with two mutations, 66 (57%) had subclones. Subclones showed significantly lower growth rates than distinct clones (β=−0.15, P=0.02) (FIG. 1E). This finding was robust for multiple definitions of distinct versus subclones (standard deviation thresholds ranging from 0.4 to 0.8). Co-occurrence analysis of CHIP mutations by driver gene (FIG. 1F) revealed that JAK2 and SF3B1/SRSF2 co-occurred significantly more often than expected (OR=48.7, p=0.0006). Two mutations in TET2 (OR=6.6, p=0.001), DNMT3A (OR=3.8, p=0.006), and PPM1D (OR=8.7, p=0.02) also occurred more frequently than chance. Conversely, DNMT3A and TET2 co-occurred less often than expected based on frequency (OR=0.4, p=0.02) (FIG. 1G).

### Example 4: Driver Gene Mutations

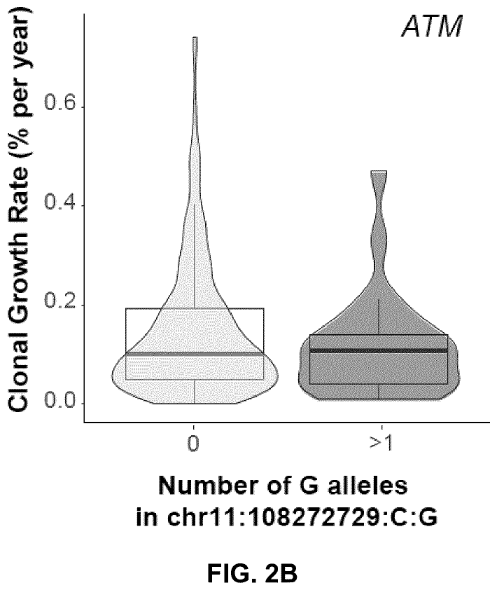

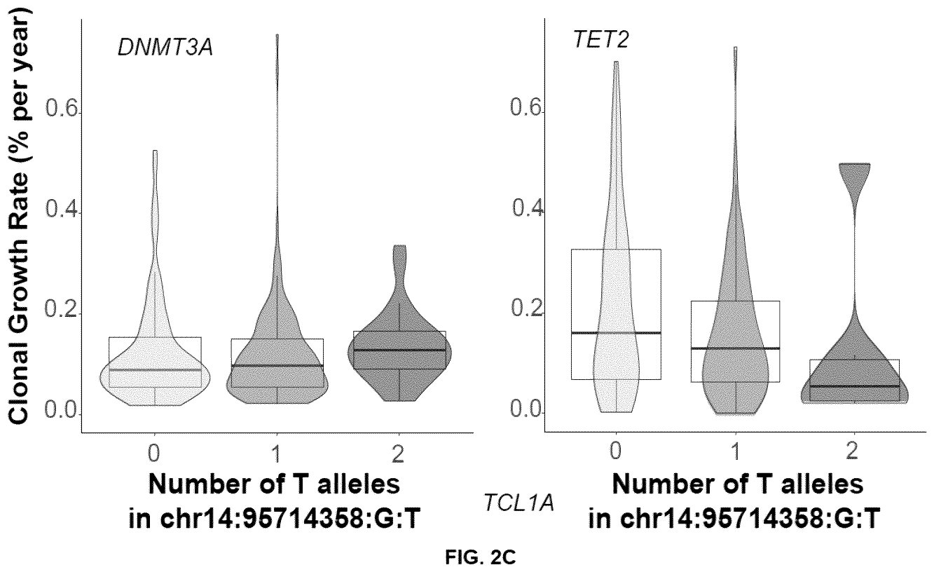

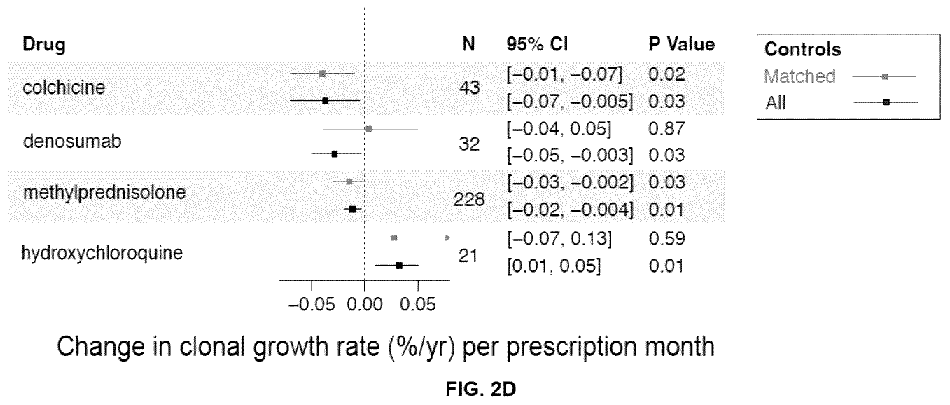

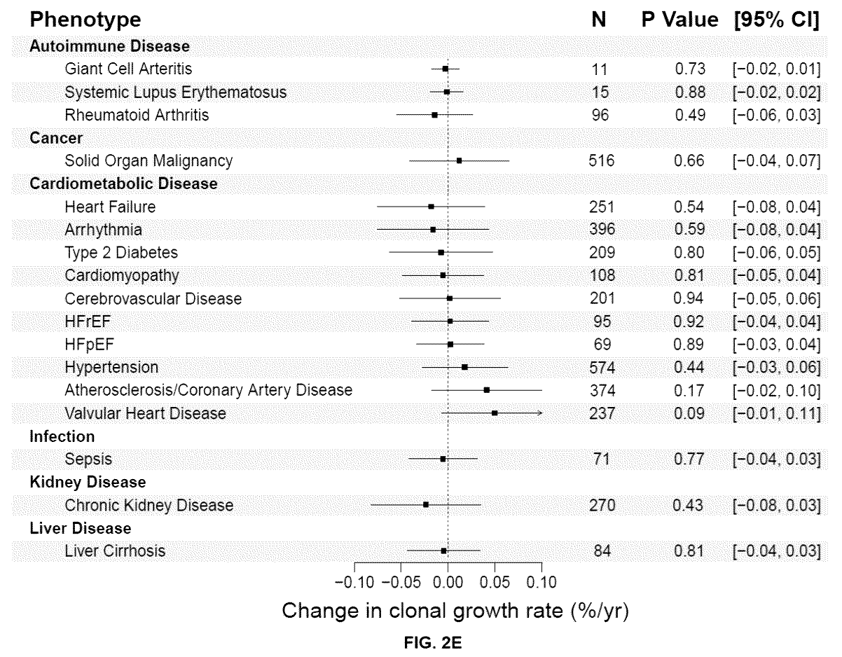

Individual determinants of CHIP expansion rate were investigated. When accounting for driver gene mutations, growth rate showed no significant association with age (P=0.07), body mass index (P=0.68), biological sex (P=0.89), or smoking status (P=0.70). The targeting assay included probes for germline variants previously linked to CHIP prevalence and estimated growth rate (FIG. 2A). Each additional G allele in rs1800057 (ATM) correlated with increased growth rate (β=0.46, 95% CI: 0.23 to 0.67, P<0.001, FIG. 2B), potentially explaining its association with CHIP prevalence. 1 Consistent with NHLBI's TOPMed cohort findings, each T allele at rs2887399 (in the TCL1A promoter) associated with increased DNMT3A expansion rate (P=0.04) and decreased TET2 expansion rate (P=0.02) (FIG. 2C).

### Example 5: Medication Exposure Duration and CHIP Growth Rate

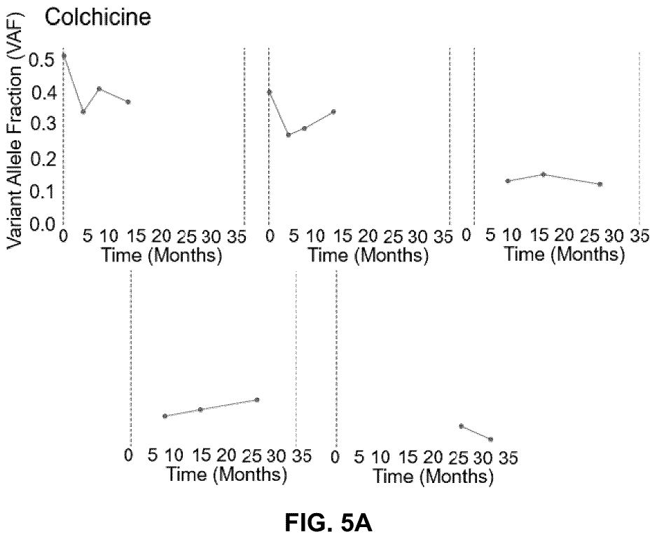

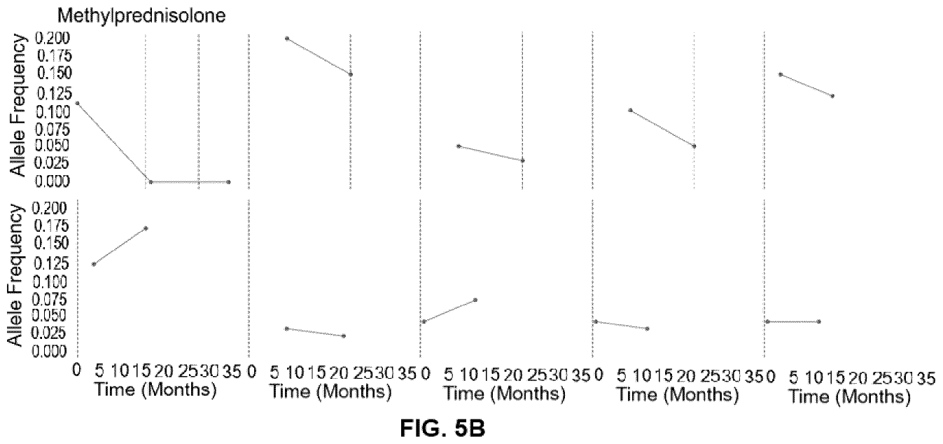

Associations between medication exposure duration and CHIP growth rate were investigated. While no medications showed significant associations after multiple hypothesis correction, several demonstrated suggestive effects (FIG. 2D). Three drugs suggested reduced annual growth rates: colchicine (β=−3.5%, 95% CI: −0.5% to −7%, P=0.03), denosumab (β=−2.5%, 95% CI: −0.3% to −5%, P=0.03), and methylprednisolone (β=−1.0%, 95% CI: −0.4% to −2%, P=0.01). These associations have biological plausibility: methylprednisolone has anti-inflammatory effects, colchicine may abrogate cardiovascular risk in TET2 CHIP, and denosumab inhibits RANKL, which DNMT3A-mutated monocytes overexpress in bone. Conversely, hydroxychloroquine suggested increased annual growth rate (β=3.1%, 95% CI: −0.3% to −5%, P=0.03). Moreover, 3:1 case-control matching was performed by age, sex, CHIP driver gene, baseline VAF, and drug indication for each drug and tested the association with growth rate. In this sensitivity analysis, an association between exposure duration between sequencing timepoints of colchicine and methylprednisolone and decreased growth rate was observed. In external validation cohort Clonal Hematopoiesis and Inflammation in the VasculaturE (CHIVE), 4/5 mutations exposed to colchicine and 8/10 exposed to methylprednisolone showed reduced or unchanged VAF (FIGS. 5A-5B). No associations between pre-existing diagnoses and CHIP expansion rate were found (FIG. 2E). The lack of association between preexisting atherosclerosis and CHIP growth rate supports a unidirectional association between clonal hematopoiesis and atherosclerosis.

### Example 6: Consequences of CHIP Expansion Rate

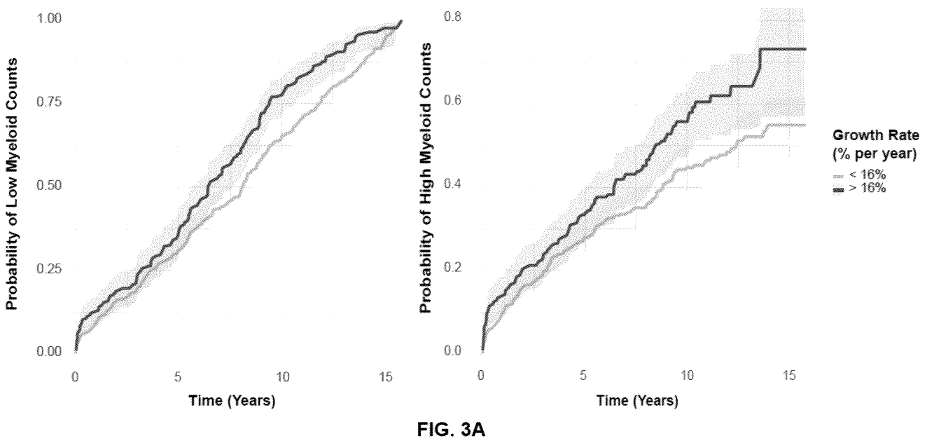

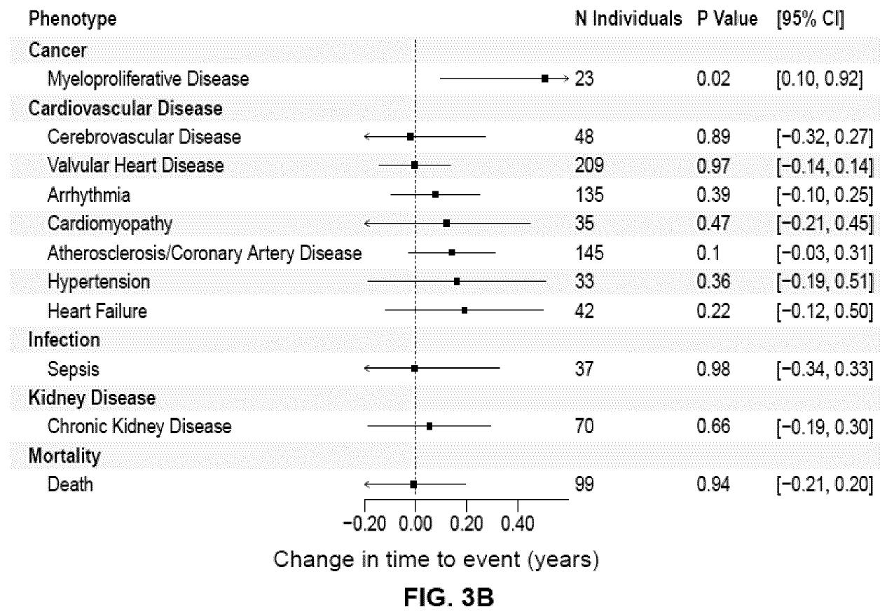

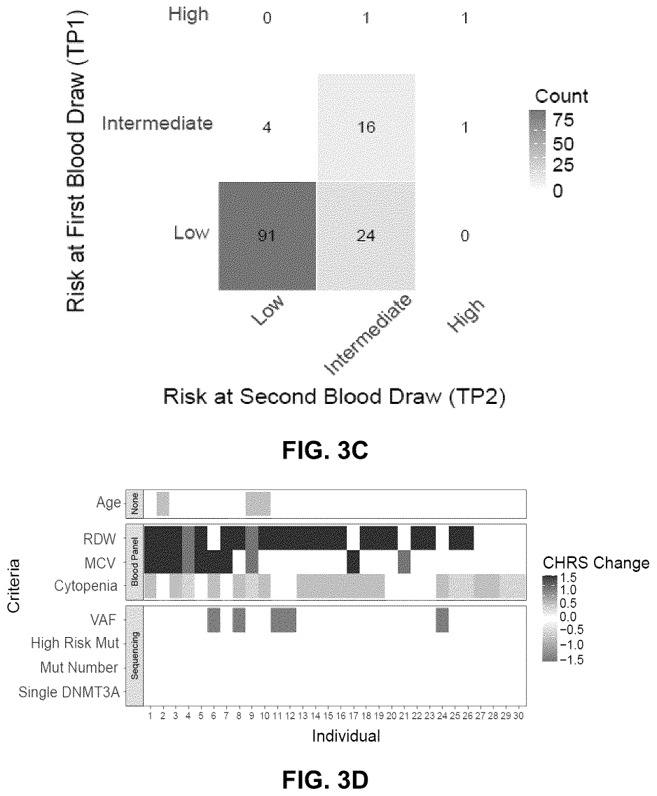

The consequences of CHIP expansion rate on blood counts and diagnoses after the second blood draw were investigated. Analyzing associations between growth rate and time to blood cell count abnormalities (as per Niroula et al.), it was found that rank-inverse normalized growth rate, adjusted for age, initial VAF, and sex, were associated with shorter time to high myeloid cell parameters (HR=1.14, 95% CI 1.02-1.27) and low myeloid cell parameters (HR=1.20, 95% CI 1.05-1.36). No associations were found with lymphocytosis (P=0.48) or lymphopenia (P=0.53). Annual growth rate >16% increased risk of both high and low myeloid cell parameters (P=0.003 and P<0.001, respectively) (FIG. 3A). A 1% increase in annual growth rate suggested a 1.6-fold increased risk of myeloproliferative disorders (95% CI: 1.1-2.5, P=0.02), though this was not significant after multiple-hypothesis correction (FIG. 3B). These findings indicate that growth rate signals heightened disease risk in individuals with CHIP.

### Example 7: Changes in Clinical Risk Scores

Changes in clinical risk scores over time were next examined, focusing on the Clonal Hematopoiesis Risk Score (CHRS) for 138 individuals with complete blood count data within 6 months of sequencing. Of 115 individuals initially classified as low risk (CHRS≤9.5), 24 (21%) were reclassified as intermediate risk (10<CHRS<12) at the second blood draw (FIG. 3C). Among 21 initially intermediate-risk individuals, 4 (19%) were reclassified as low risk, and 1 (5%) as high risk (CHRS≥12.5). Only 2 individuals were classified as high risk overall. Risk category changes were primarily driven by alterations in blood counts (FIG. 3D). All individuals with CHRS category changes showed alterations in RDW, MCV, or blood counts, while only 5 (17%) exceeded the 20% VAF threshold, all of whom also had blood count changes. These findings suggest that repeated blood sequencing may be unnecessary without accompanying laboratory abnormalities.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

The following examples are put forth so as to pr
1. Bick A G, et al. Inherited causes of clonal haematopoiesis in 97,691 whole genomes. Nature 2020; 586 (7831): 763-768.
2. Díez-Díez M, et al. Unidirectional association of clonal hematopoiesis with atherosclerosis development. Nat Med 2024; 30:2857-2866.
3. Fabre M A, et al. The longitudinal dynamics and natural history of clonal haematopoiesis. Nature 2022; 606 (7913): 335-342.
4. Jaiswal S, et al. Age-Related Clonal Hematopoiesis Associated with Adverse Outcomes. N Engl J Med 2014; 371 (26): 2488-2498.
5. Jaiswal S, et al. Clonal Hematopoiesis and Risk of Atherosclerotic Cardiovascular Disease. New England Journal of Medicine 2017; 377 (2): 111-121.
6. Kessler M D, et al. Common and rare variant associations with clonal haematopoiesis phenotypes. Nature 2022; 612 (7939): 301-309.
7. Mack T, et al. Cost-effective and scalable clonal hematopoiesis assay provides insight into clonal dynamics. J Mol Diagn 2024; 26 (7): 563-573.
8. Niroula A, et al. Distinction of lymphoid and myeloid clonal hematopoiesis. Nat Med 2021; 27 (11): 1921-1927.
9. Shannon M L, et al. Clonal Hematopoiesis and Inflammation in the VasculaturE (CHIVE): a prospective, longitudinal cohort and biorepository. Blood Advances 2024; 8 (13): 3453-3463.
10. Uddin M M, et al. Longitudinal profiling of clonal hematopoiesis provides insight into clonal dynamics. Immun Ageing 2022; 19 (1): 23.
11. Vlasschaert C, et al. A practical approach to curate clonal hematopoiesis of indeterminate potential in human genetic data sets. Blood 2023; 141 (18): 2214-2223.
12. Wang H. Clonal hematopoiesis driven by mutated DNMT3A promotes inflammatory bone loss. Cell 2024; (187): 1-22.
13. Weeks L D, et al. Prediction of Risk for Myeloid Malignancy in Clonal Hematopoiesis. NEJM Evidence; 2023; 2 (5).
14. Weinstock J S, et al. Aberrant activation of TCL1A promotes stem cell expansion in clonal hematopoiesis. Nature 2023; 616 (7958): 755-763.

What is claimed is:

1. A method for treating clonal hematopoiesis of indeterminate potential (CHIP) in a subject, the method comprising administering to the subject a therapeutically effective amount of an inhibitor of receptor activator of nuclear factor κB ligand (RANKL), a therapeutically effective amount of an anti-inflammatory agent, or both.

2. The method of claim 1, wherein the inhibitor of RANKL comprises denosumab.

3. The method of claim 2, wherein the inhibitor of RANKL is administered by injection.

4. The method of claim 2, wherein the inhibitor of RANKL is administered once every six months.

5. The method of claim 1, wherein the anti-inflammatory agent comprises colchicine.

6. The method of claim 5, wherein the anti-inflammatory agent is administered orally.

7. The method of claim 5, wherein the anti-inflammatory agent is administered daily.

8. The method of claim 1, further comprising detecting at least one CHIP mutation in the subject prior to performing the method.

9. The method of claim 1, wherein performing the method reduces growth of one or more clones relative to an untreated control.

10. The method of claim 1, wherein performing the method causes a size of one or more clones to remain constant.

11. The method of claim 1, wherein the method is performed for at least one year.

12. The method of claim 11, wherein each month of performing the method results in a reduction of annual clonal growth rate of from about 2.5% to about 3.5%.

13. The method of claim 1, wherein both an inhibitor of RANKL and an anti-inflammatory agent are administered.

14. The method of claim 1, wherein performing the method further treats or prevents at least one cardiometabolic or cardiovascular disease.

15. The method of claim 14, wherein the at least one cardiometabolic or cardiovascular disease comprises peripheral artery disease, non-alcoholic fatty liver disease (NAFLD), hypertension, hyperlipidemia, or atherosclerosis.

16. The method of claim 1, wherein performing the method further treats or prevents at least one myeloproliferative disease or disorder.

17. The method of claim 16, wherein the at least one myeloproliferative disease or disorder comprises chronic myelomonocytic leukemia, chronic myeloid leukemia, anemia, thrombocytopenia, thrombocytosis, polycythemia, or any combination thereof.

18. A method for reducing or maintaining clonal hematopoiesis risk score (CHRS) in a subject, the method comprising administering to the subject a therapeutically effective amount of an inhibitor of receptor activator of nuclear factor κB ligand (RANKL), a therapeutically effective amount of an anti-inflammatory agent, or both.

19. The method of claim 18, wherein the inhibitor of RANKL comprises denosumab.

20. The method of claim 18, wherein the anti-inflammatory agent comprises colchicine.
